(19) United States
(12) Patent Application Publication
(10) Pub. No.: US 2013/0281308 A1
(43) Pub. Date: Oct. 24, 2013

(54) Methods for sorting nucleic acids and preparative in vitro cloning

(71) Applicant: GEN9, INC., Cambridge, MA (US)

(72) Inventors: Li-yun A. Kung, Arlington, MA (US); Joseph Jacobson, Newton, MA (US)

(21) Appl. No.: 13/986,368

(22) Filed: Apr. 24, 2013

Related U.S. Application Data
(60) Provisional application No. 61/637,750, filed on Apr. 24, 2012.
(60) Provisional application No. 61/638,187, filed on Apr. 25, 2012.
(60) Provisional application No. 61/731,626, filed on Nov. 30, 2012.

(51) Int. Cl. C12N 15/10

(52) U.S. Cl. CPC C12N 15/1065
USPC 506/4

(57) ABSTRACT

Methods and compositions relate to the sorting and cloning of high fidelity nucleic acids using high throughput sequencing. Specifically, nucleic acid molecules having the desired predetermined sequence can be sorted from a pool comprising a plurality of nucleic acids having correct and incorrect sequences.

### RELATED APPLICATIONS

This application claims the benefit of and priority to U.S. provisional application Ser. No. 61/637,750, filed Apr. 24, 2012, U.S. provisional application Ser. No. 61/638,187, filed Apr. 25, 2012, and U.S. provisional application Ser. No. 61/731,626, filed Nov. 30, 2012, each of which is incorporated herein by reference in its entirety.

### STATEMENT REGARDING FEDERALLY SPONSORED RESEARCH

This invention was made with United States Government support under the cooperative agreement number 70NANB7H7034N awarded by the National Institute of Standards and Technology. The United States Government has certain rights in the invention.

### FIELD OF THE INVENTION

Methods and compositions of the invention relate to nucleic acid assembly, and particularly to methods for sorting and cloning nucleic acids having a predetermined sequence.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids also can be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids may also be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of currently available assembly techniques is the relatively high error rate. As such, high fidelity, low cost assembly methods are needed.

### SUMMARY OF THE INVENTION

Aspects of the invention relate to methods of sorting and cloning nucleic acid molecules having a desired or predetermined sequence. In some embodiments, the method comprises contacting a pool of nucleic acid molecules comprising at least two populations of nucleic acid molecules, each population of nucleic acid molecules having a unique nucleic acid sequence, tagging the 5′ end and the 3′ end of the nucleic acid molecules with a oligonucleotide tag sequence, wherein the oligonucleotide tag sequence comprises a unique nucleotide tag and a primer region, subjecting the nucleic acid molecules to sequencing reactions from both ends to obtain paired end reads, and sorting the nucleic acid molecules having the desired sequence according to the identity of their corresponding unique nucleotide tags. In some embodiments, each population of nucleic acid molecules has a different desired nucleic acid sequence. In some embodiments, the unique nucleotide tag is ligated or joined at each end of the nucleic acid molecules by PCR. In some embodiments, the unique nucleotide tag has a degenerate sequence.

In some embodiments, the method further comprises amplifying the nucleic acid molecules having the desired sequence. In some embodiments, the method comprises amplifying the constructs having the desired sequence using primers complementary to the primer region and the tag sequence.

In some embodiments, the method further comprises pooling a plurality of nucleic acid molecules to form the pool of nucleic acid molecules, wherein each plurality of nucleic acid molecules comprises a population of nucleic acid sequence having the desired sequence and a population of nucleic acid having a sequence different than the desired sequence. In some embodiments, the nucleic acid molecules can be assembled de novo. In some embodiments, the plurality of nucleic acid molecules can be diluted prior to the step of pooling or after the step of pooling to form a normalized pool of nucleic acid molecules.

In some embodiments, each nucleic acid molecule comprises a 5′ end common adaptor sequence and 3′ end common adaptor sequence and the oligonucleotide tag sequence further comprises a common adaptor sequence.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A illustrates steps I, II, and III of a non-limiting exemplary method of preparative cloning according to some embodiments. FIG. 1B illustrates steps IV and V of a non-limiting exemplary method of preparative cloning according to some embodiments. FIG. 1C illustrates the preparative recovery of correct clones, step VI, of a non-limiting exemplary method of preparative cloning according to some embodiments.

### DETAILED DESCRIPTION OF THE INVENTION

Techniques have been developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids. However, one limitation of currently available assembly techniques is the relatively high error rate. There is therefore a need to isolate nucleic acid constructs having a predetermined sequence and discarding constructs having nucleic acid errors.

Aspects of the invention can be used to isolate nucleic acid molecules from large numbers of nucleic acid fragments efficiently, and/or to reduce the number of steps required to generate large nucleic acid products, while reducing error rate. Aspects of the invention can be incorporated into nucleic assembly procedures to increase assembly fidelity, throughput and/or efficiency, decrease cost, and/or reduce assembly time. In some embodiments, aspects of the invention may be automated and/or implemented in a high throughput assembly context to facilitate parallel production of many different target nucleic acid products. In some embodiments, nucleic acid constructs may be assembled using starting nucleic acids obtained from one or more different sources (e.g., synthetic or natural polynucleotides, nucleic acid amplification products, nucleic acid degradation products, oligonucleotides, etc.).

As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to about 250, about 40 to about 220, about 50 to about 200, about 60 to about 180, or about 65 or about 150 nucleotides long), between about 100 and about 200 nucleotides long, between about 200 and about 300 nucleotides long, between about 300 and about 400 nucleotides long, or between about 400 and about 500 nucleotides long. However, shorter or longer oligonucleotides may be used. An oligonucleotide may be a single-stranded or double-stranded nucleic acid. As used herein the terms “nucleic acid”, “polynucleotide”, “oligonucleotide” are used interchangeably and refer to naturally-occurring or synthetic polymeric forms of nucleotides. The oligonucleotides and nucleic acid molecules of the present invention may be formed from naturally occurring nucleotides, for example forming deoxyribonucleic acid (DNA) or ribonucleic acid (RNA) molecules. Alternatively, the naturally occurring oligonucleotides may include structural modifications to alter their properties, such as in peptide nucleic acids (PNA) or in locked nucleic acids (LNA). The solid phase synthesis of oligonucleotides and nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the invention include, for example, naturally-occurring nucleotides (for example, ribonucleotides or deoxyribonucleotides), or natural or synthetic modifications of nucleotides, or artificial bases. As used herein, the term monomer refers to a member of a set of small molecules which are and can be joined together to form an oligomer, a polymer or a compound composed of two or more members. The particular ordering of monomers within a polymer is referred to herein as the “sequence” of the polymer. The set of monomers includes, but is not limited to for example, the set of common L-amino acids, the set of D-amino acids, the set of synthetic and/or natural amino acids, the set of nucleotides and the set of pentoses and hexoses. Aspects of the invention described herein primarily with regard to the preparation of oligonucleotides, but could readily be applied in the preparation of other polymers such as peptides or polypeptides, polysaccharides, phospholipids, heteropolymers, polyesters, polycarbonates, polyureas, polyamides, polyethyleneimines, polyarylene sulfides, polysiloxanes, polyimides, polyacetates, or any other polymers.

In some embodiments, each nucleic acid fragment or construct (also referred herein as target nucleic acid) being assembled may be between about 100 nucleotides long and about 1,000 nucleotides long (e.g., about 200 nucleotides long, about 300 nucleotides long, about 400 nucleotides long, about 500 nucleotides long, about 600 nucleotides long, about 700 nucleotides long, about 800 nucleotides long, about 900 nucleotides long). However, longer (e.g., about 2,500 or more nucleotides long, about 5,000 or more nucleotides long, about 7,500 or more nucleotides long, about 10,000 or more nucleotides long, etc.) or shorter nucleic acid fragments may be assembled using an assembly technique (e.g., shotgun assembly into a plasmid vector). It should be appreciated that the size of each nucleic acid fragment may be independent of the size of other nucleic acid fragments added to an assembly. However, in some embodiments, each nucleic acid fragment may be approximately the same size.

Aspects of the invention relate to methods and compositions for the selective isolation of nucleic acid constructs having a predetermined sequence of interest. As used herein, the term “predetermined sequence” means that the sequence of the polymer is known and chosen before synthesis or assembly of the polymer. In particular, aspects of the invention is described herein primarily with regard to the preparation of nucleic acids molecules, the sequence of the oligonucleotide or polynucleotide being known and chosen before the synthesis or assembly of the nucleic acid molecules. In some embodiments of the technology provided herein, immobilized oligonucleotides or polynucleotides are used as a source of material. In various embodiments, the methods described herein use pluralities of oligonucleotides, each sequence being determined based on the sequence of the final polynucleotides constructs to be synthesized. In one embodiment, oligonucleotides are short nucleic acid molecules. For example, oligonucleotides may be from 10 to about 300 nucleotides, from 20 to about 400 nucleotides, from 30 to about 500 nucleotides, from 40 to about 600 nucleotides, or more than about 600 nucleotides long. However, shorter or longer oligonucleotides may be used. Oligonucleotides may be designed to have different length. In some embodiments, the sequence of the polynucleotide construct may be divided up into a plurality of shorter sequences that can be synthesized in parallel and assembled into a single or a plurality of desired polynucleotide constructs using the methods described herein.

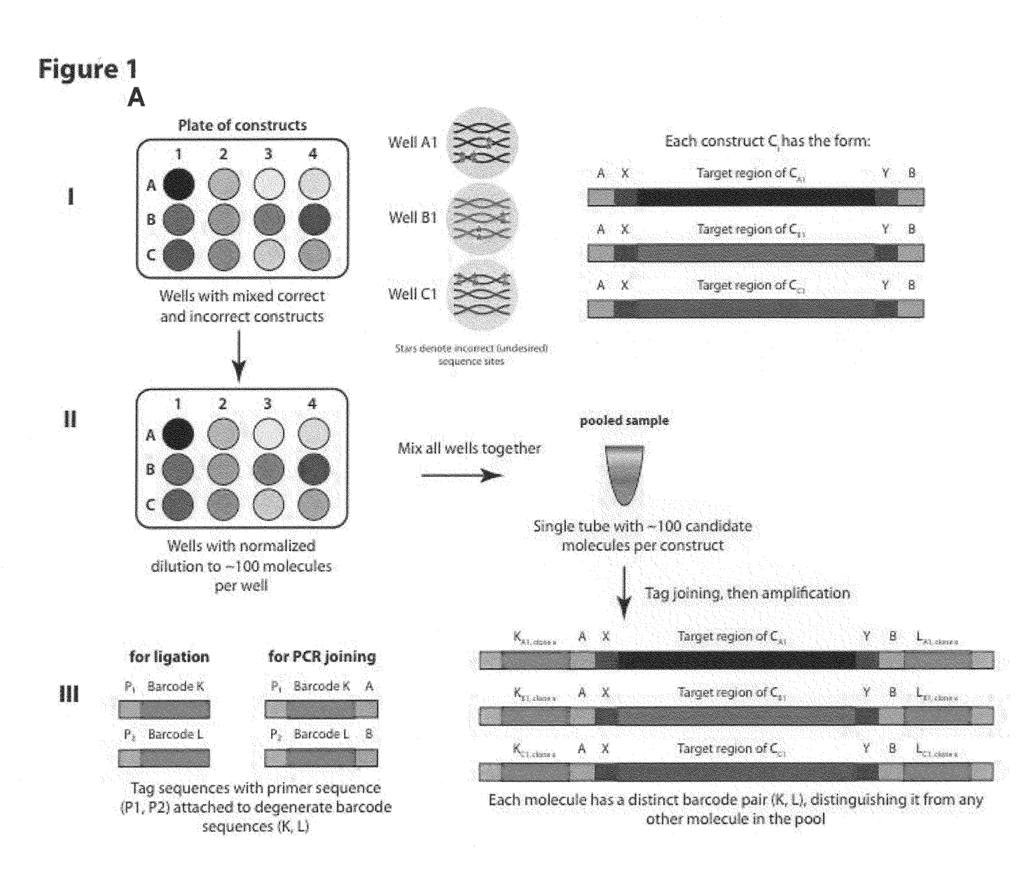

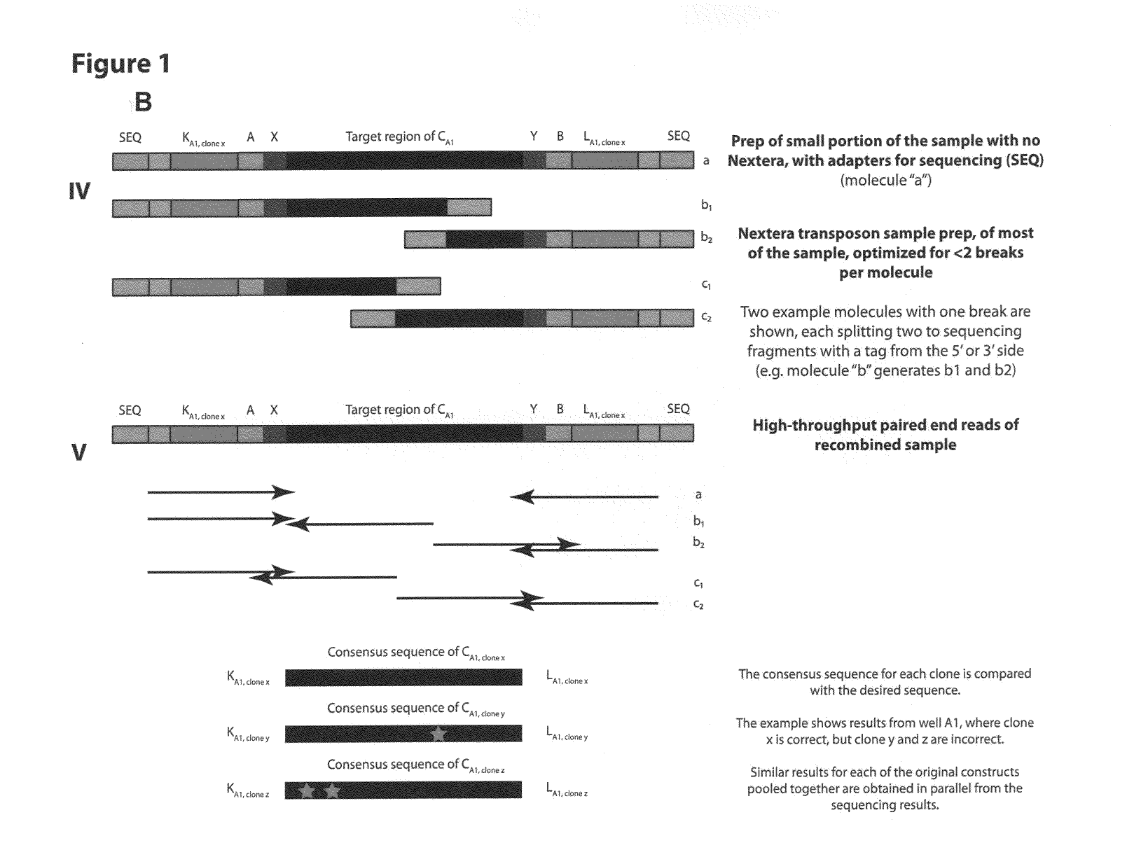

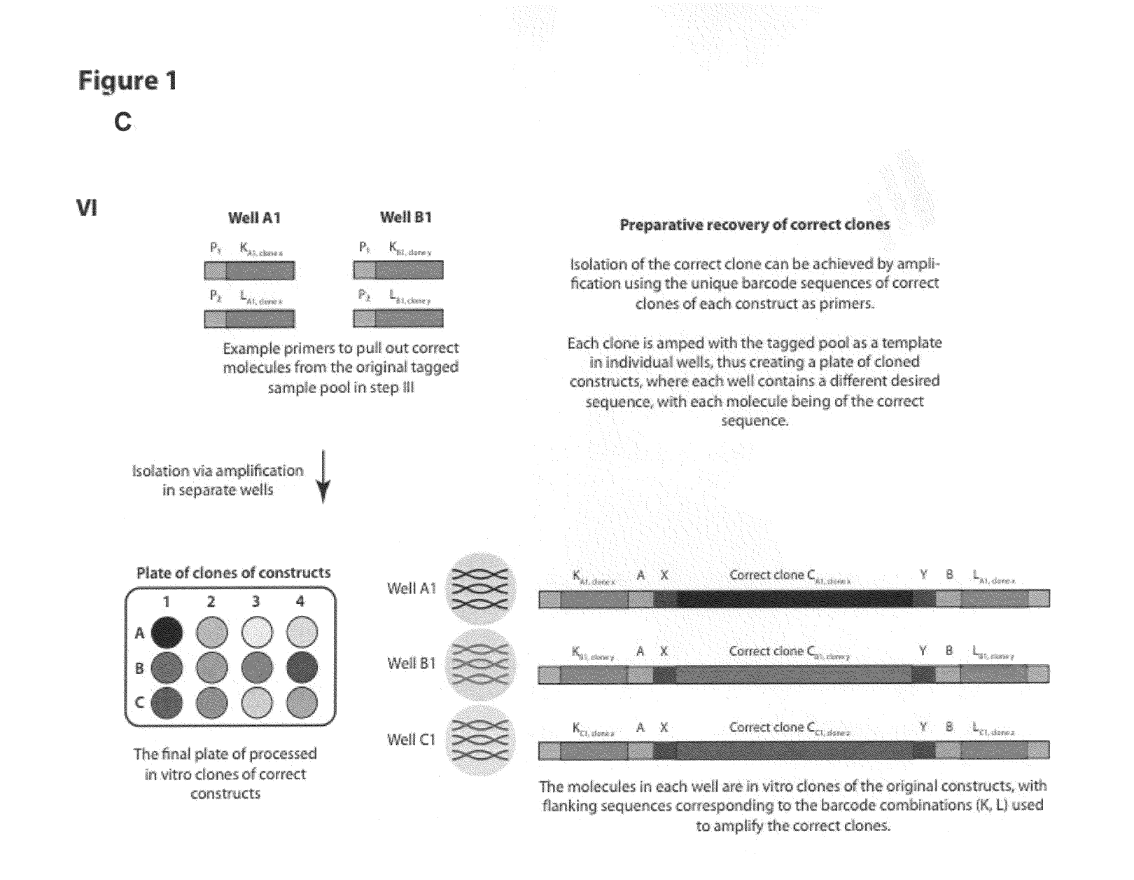

In some embodiments, the methods described herein allow for the cloning of nucleic acid sequences having a desired or predetermined sequence from a pool of nucleic acid molecules. In some embodiments, the methods may include analyzing the sequence of target nucleic acids for parallel preparative cloning of a plurality of target nucleic acids. For example, the methods described herein can include a quality control step and/or quality control readout to identify the nucleic acid molecules having the correct sequence. FIGS. 1A-C show an exemplary method for isolating and cloning nucleic acid molecules having predetermined sequences. In some embodiments, the nucleic acid can be first synthesized or assembled onto a support. For example, the nucleic acid molecules can be assembled in a 96-well plate with one construct per well. In some embodiments, each nucleic acid construct (C₁ through C_{N}, FIGS. 1A-C) has a different nucleotide sequence. For example, the nucleic acid constructs can be non-homologous nucleic acid sequences or nucleic acid sequences having a certain degree of homology. Yet in other embodiments, a plurality of nucleic acid molecules having a predefined sequence, e.g. C₁ through C_{N}, can be deposited at different locations or wells of a solid support. In some embodiments, the limit of the length of the nucleic acid constructs can depend on the efficiency of sequencing the 5′ end and the 3′ end of the full length target nucleic acids via high-throughput paired end sequencing. One skilled in the art will appreciate that the methods described herein can bypass the need for cloning via the transformation of cells with nucleic acid constructs in propagatable vectors. In addition, the methods described herein eliminate the need to amplify candidate constructs separately before identifying the target nucleic acids having the desired sequences.

One skilled in the art would appreciate that after oligonucleotide assembly, the assembly product may contain a pool of sequences containing correct and incorrect assembly products. For example, referring to FIGS. 1A-C, each well of the plate (nucleic acid construct C₁ through C_{N}) can be a mixture of nucleic acid molecules having correct or incorrect sequences. The errors may result from sequence errors introduced during the oligonucleotide synthesis, or during the assembly of oligonucleotides into longer nucleic acids. In some instances, up to 90% of the nucleic acid sequences may be unwanted sequences. Devices and methods to selectively isolate the correct nucleic acid sequence from the incorrect nucleic acid sequences are provided herein. The correct sequence may be isolated by selectively isolating the correct sequence from the other incorrect sequences as by selectively moving or transferring the desired assembled polynucleotide of predefined sequence to a different feature of the support, or to another plate. Alternatively, polynucleotides having an incorrect sequence can be selectively removed from the feature comprising the polynucleotide of interest. According to some methods of the invention, the assembly nucleic acid molecules may first be diluted within the solid support in order to obtain a normalized population of nucleic acid molecules. As used herein, the term “normalized” or “normalized pool” means a nucleic acid pool that has been manipulated, to reduce the relative variation in abundance among member nucleic acid molecules in the pool to a range of no greater than about 1000-fold, no greater than about 100-fold, no greater than about 10-fold, no greater than about 5-fold, no greater than about 4-fold, no greater than about 3-fold or no greater than about 2-fold. In some embodiments, the nucleic acid molecules are normalized by dilution. For example, the nucleic acid molecules can be normalized such as the number of nucleic acid molecules is in the order of about 5, about 10, about 20, about 30, about 40, about 50, about 60, about 60, about 70, about 80, about 90, about 100, about 1000 or higher. In some embodiments, each population of nucleic acid molecules can be normalized by limiting dilution before pooling the nucleic acid molecules to reduce the complexity of the pool. In some embodiments, to ensure that at least one copy of the target nucleic acid sequence is present in the pool, dilution can be limited to provide for more than one nucleic acid molecule. In some embodiments, the oligonucleotides can be diluted serially. In some embodiments, the device (for example, an array or microwell plate, such as 96 wells plate) can integrate a serial dilution function. In some embodiments, the assembly product is serially diluted to a produce a normalized population of nucleic acids. In some embodiments, the concentration and the number of molecules can be assessed prior to the dilution step and a dilution ratio can be calculated in order to produce a normalized population. In an exemplary embodiment, the assembly product is diluted by a factor of at least 2, at least 3, at least 4, at least 5, at least 6, at least 7, at least 10, at least 20, at least 50, at least 100, at least 1,000 etc. . . . In some embodiments, prior to sequencing, the target nucleic acid sequences can be diluted and placed, for example, in distinct wells or at distinct locations of a solid support or on distinct supports.

In some embodiments, the normalized populations of nucleic acid molecules can be pooled to create a pool of nucleic acid molecules having different predefined sequences. In some embodiments, each nucleic acid molecule in the pool can be at a relatively low complexity. Yet in other embodiments, normalization of the nucleic acid molecules can be performed after mixing the different population of nucleic acid molecules present at high concentration.

In some embodiments, the 5′ end and the 3′ end of each nucleic acid molecules within the pool can be tagged with a pair of tag oligonucleotide sequence. In some embodiments, the tag oligonucleotide sequence can be composed of common DNA primer regions and unique “barcode” regions such as a specific nucleotide sequence. In some embodiments, the number of tag nucleotide sequences can be greater than the number of molecules per construct (i.e. 10-1000 molecules in the dilution). For example, a 6 bp, 7 bp, 8 bp, or longer nucleotide tag can be used. In some embodiments, a NNNNNNNN (8 degenerate bases) can be used and generates 65,536 unique barcodes. In some embodiments, the length of the nucleotide tag can be chosen such as to limit the number of pairs of tags that share a common tag sequence for each nucleic acid construct.

In some embodiments, the tag oligonucleotide sequences can be joined to each nucleic acid molecule to form a nucleic acid molecule comprising a tag oligonucleotide sequence at its 5′ and 3′ ends. In some embodiments, the tag oligonucleotide sequences can be ligated to a blunt end nucleic acid molecule using a ligase. For example, the ligase can be a T7 ligase or any other ligase capable of ligating the tag oligonucleotide sequences to the nucleic acid molecules. Preferably, ligation is performed under conditions suitable to avoid concatamerization of the nucleic acid constructs. In other embodiments, the nucleic acid molecules are designed to have at their 5′ and 3′ ends a sequence that is common or complementary to the tag oligonucleotide sequences. In some embodiments, the tag oligonucleotide sequences and the nucleic acid molecules having common sequences can be joined as adaptamers by polymerase chain reaction.

In some embodiments, the target nucleic acid sequence or a copy of the target nucleic acid sequence can be isolated from a pool of nucleic acid sequences, some of them containing one or more sequence errors. As used herein, a copy of the target nucleic acid sequence refers to a copy using template dependent process such as PCR. In some embodiments, sequence determination of the target nucleic acid sequences can be performed using sequencing of individual molecules, such as single molecule sequencing, or sequencing of an amplified population of target nucleic acid sequences, such as polony sequencing. In preferred embodiments, the pool of nucleic acid molecules is subjected to high throughput paired end sequencing reactions, such as using the HiSeq, MiSeq (Illumina) or the like.

In some embodiments, the nucleic acid molecules are amplified using the common primer sequences on each tag oligonucleotide sequence. In some embodiments, the primer can be universal primers or unique primer sequences. Amplification allows for the preparation of the target nucleic acids for sequencing, as well as to retrieve the target nucleic acids having the desired sequences after sequencing. In some embodiments, a sample of the nucleic acid molecules is subjected to transposon-mediated fragmentation and adapter ligation to enable rapid preparation for paired end reads using high throughput sequencing systems. For example, the sample can be prepared to undergo Nextera™ tagmentation (Illumina).

On skilled in the art will appreciate that it can be important to control the extent of the fragmentation and the size of the nucleic acid fragments to maximize the number of reads in the sequencing paired reads and thereby allow for sequencing the desired length of the fragment. In some embodiments, the paired end reads can generate one sequence with a tag for identification, and another sequence which is internal to the construct target region. With high throughput sequencing, enough coverage can be generated to reconstruct the consensus sequence of each tag pair construct and determine if the construct sequence is correct. In some embodiments, it is preferable to limit the number of breakage to less than 2, less than 3, or less than 4. In some embodiments the extent of the fragmentation and/or the size of the fragments can be controlled using appropriate reaction conditions such as by using the suitable concentration of transposon enzyme and controlling the temperature and time of incubation. Suitable reaction conditions can be obtained by using known amounts of a test library and titrating the enzyme and time to build a standard curve for actual sample libraries. In some embodiments, a portion of the sample, which is not used for fragmentation, can be mixed back into the fragmented sample and processed for sequencing.

The sample can then sequenced on a platform that generates paired end reads. Depending on the size of the individual DNA constructs, the number of constructs mixed together, and the estimated error rate of the populations, the appropriate platform can be chosen to maximize the number of reads desired and minimize the cost per construct.

The sequencing of the nucleic acid molecules results in reads with both of the tags from each molecule in the paired end reads. The paired end reads can be used to identify which pairs of tags were ligated or PCR joined and the identity of the molecule.

For data analysis, reads for which one tag is paired with multiple other tags for the same construct are discarded, because this would result in ambiguity as to which clone the data came from.

The sequencing results can then be analyzed to determine the sequences of each clone of each construct. For each paired read where one read contains a tag sequence, the identity of the molecule each sequencing read comes from is known, and the construct sequence itself can be used to distinguish between constructs with the same tag. The other read from the paired read can be used to build a consensus sequence of the internal regions of the molecule. From these results, a mapping of tag pairs corresponding to correct target sequence for each construct can be generated.

In some embodiments, the target having the desired sequence can be recovered using the methods for recovery of the annotated correct target sequences disclosed herein. In some embodiments, the tag sequence pairs for each correct target sequence can be used to amplify by PCR the construct from the sample pool (see FIG. 1B, step IV). It should be noted that since the likelihood of the same pair being used for multiple molecules is extremely low, the likelihood to isolate the nucleic acid molecule having the correct sequence is high. Yet in other embodiments, the nucleic acid having the desired sequence can be recovered directly from the sequencer. In some embodiments, the identity of a full length construct can be determined once the pairs of tags are identified. In principle, the location of the full length read (corresponding to a paired end read with the 5′ and 3′ tags) can be determined on the original sequencing flow cell. After locating the cluster on the flow cell surface, molecules can be eluted or otherwise captured from the surface.

### Applications

Aspects of the invention may be useful for a range of applications involving the production and/or use of synthetic nucleic acids. As described herein, the invention provides methods for producing synthetic nucleic acids having the desired sequence with increased efficiency. The resulting nucleic acids may be amplified in vitro (e.g., using PCR, LCR, or any suitable amplification technique), amplified in vivo (e.g., via cloning into a suitable vector), isolated and/or purified. An assembled nucleic acid (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, the nucleic acid may be integrated into the genome of the host cell. In some embodiments, the nucleic acid may replace a corresponding nucleic acid region on the genome of the cell (e.g., via homologous recombination). Accordingly, nucleic acids may be used to produce recombinant organisms. In some embodiments, a target nucleic acid may be an entire genome or large fragments of a genome that are used to replace all or part of the genome of a host organism. Recombinant organisms also may be used for a variety of research, industrial, agricultural, and/or medical applications.

Many of the techniques described herein can be used together, applying suitable assembly techniques at one or more points to produce long nucleic acid molecules. For example, ligase-based assembly may be used to assemble oligonucleotide duplexes and nucleic acid fragments of less than 100 to more than 10,000 base pairs in length (e.g., 100 mers to 500 mers, 500 mers to 1,000 mers, 1,000 mers to 5,000 mers, 5, 000 mers to 10,000 mers, 25,000 mers, 50,000 mers, 75,000 mers, 100,000 mers, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including nucleic acids that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the invention can be incorporated into a vector. The vector may be a cloning vector or an expression vector. In some embodiments, the vector may be a viral vector. A viral vector may comprise nucleic acid sequences capable of infecting target cells. Similarly, in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e. using cell-free systems) or in vivo (i.e. expressed in cells). In some embodiments, cell lysates may be prepared. In certain embodiments, expressed RNAs or polypeptides may be isolated or purified. Nucleic acids of the invention also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP₅ GST, MBP, chitin and the like. In some embodiments, polypeptides may comprise one or more unnatural amino acid residue(s).

In some embodiments, antibodies can be made against polypeptides or fragment(s) thereof encoded by one or more synthetic nucleic acids. In certain embodiments, synthetic nucleic acids may be provided as libraries for screening in research and development (e.g., to identify potential therapeutic proteins or peptides, to identify potential protein targets for drug development, etc.) In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

### Automated Applications

Aspects of the methods and devices provided herein may include automating one or more acts described herein. In some embodiments, one or more steps of an amplification and/or assembly reaction may be automated using one or more automated sample handling devices (e.g., one or more automated liquid or fluid handling devices). Automated devices and procedures may be used to deliver reaction reagents, including one or more of the following: starting nucleic acids, buffers, enzymes (e.g., one or more ligases and/or polymerases), nucleotides, salts, and any other suitable agents such as stabilizing agents. Automated devices and procedures also may be used to control the reaction conditions. For example, an automated thermal cycler may be used to control reaction temperatures and any temperature cycles that may be used. In some embodiments, a scanning laser may be automated to provide one or more reaction temperatures or temperature cycles suitable for incubating polynucleotides. Similarly, subsequent analysis of assembled polynucleotide products may be automated. For example, sequencing may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g., a robotic system) or in a micro-environment (e.g., a micro-fluidic reaction chamber). Assembly reaction mixtures (e.g., liquid reaction samples) may be transferred from one component of the system to another using automated devices and procedures (e.g., robotic manipulation and/or transfer of samples and/or sample containers, including automated pipetting devices, micro-systems, etc.). The system and any components thereof may be controlled by a control system.

Accordingly, method steps and/or aspects of the devices provided herein may be automated using, for example, a computer system (e.g., a computer controlled system). A computer system on which aspects of the technology provided herein can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the technology provided herein are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the technology provided herein. Connections between the different components of the system may be via wire, optical fiber, wireless transmission, satellite transmission, any other suitable transmission, or any combination of two or more of the above.

Each of the different aspects, embodiments, or acts of the technology provided herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the technology provided herein comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the technology provided herein. The computer-readable medium can be transportable such that the program stored thereon can be loaded onto any computer system resource to implement one or more functions of the technology provided herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the technology provided herein.

It should be appreciated that in accordance with several embodiments of the technology provided herein wherein processes are stored in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Methods for Preparative In Vitro Cloning with High Throughput Paired End Sequencing

The methods described herein and illustrated in FIGS. 1A-C allow for the identification of target nucleic acids having the correct desired sequence from a plate having a plurality of distinct nucleic acid constructs, each plurality of nucleic acid constructs comprising a mixture of correct and incorrect sequences.

In step I, FIG. 1A, a plurality of constructs (C_{A1}-C_{An}, . . . C_{N1}-C_{Nn}) is provided within separate wells of a microplate, each well comprising a mixture of correct and incorrect sequence sites. Each construct can have a target region flanked at the 5′ end with a construct specific region X and a common region or adaptor A and at the 3′ end a construct specific region Y and a common region or adaptor B.

In step II, FIG. 1A, each of the construct mixture can be diluted to a limited number of molecules (about 100-1000) such as each well of the plate comprises a normalized mixture of molecules. Each of the dilutions can be mixed and pooled together into one tube.

In step III, FIG. 1A, the plurality of molecules is tagged with pairs of primers (P1, P2) and a large library of nucleotide tags or barcodes (K,L) by ligation or polymerase chain reaction. The methods described herein allow for each molecule to be tagged with a unique pair of barcodes (K, L) to distinguish the molecule from the other molecules in the pool. For example, each well can comprise about 100 molecules and each molecule can be tagged with a unique (K, L) tag (e.g. K₁-L₁; Kⱼ-Lⱼ, . . . K₁₀₀-L₁₀₀). The entire sample can be amplified to generate enough material for sequencing and the preparative recovery.

In step IV, FIG. 1B, the sample can be split, with the bulk of the sample undergoing Nextera™ tagmentation. The tagmentation reaction can be optimized to make under two breakages per molecule, ensuring that the bulk of the molecules contain one of the tag barcodes and a partial length of the construct target region. The reserved portion of the sample that did not undergo tagmentation, is mixed back in and prepped for sequencing. Two example molecules with one break are shown, each splitting two to sequencing fragments with a tag from the 5′ or 3′ end. For example, as illustrated in FIG. 1B, molecule b can be split in two to generate b1 and b2.

In step V, FIG. 1B, the full length molecules generate paired reads which map the tag pairs (Kj, Lj) to individual clonal construct molecules (for example construct C₁, clone j in well 1). The Nextera™ tagmented paired reads generate one sequence with a tag for identification, and another sequence internal to the construct target region. With high throughput sequencing, enough coverage can be generated to reconstruct the consensus sequence of each tag pair construct and determine if the sequence correct. For example, as illustrated in FIG. 1B, each fragment in sequencing generates two reads (a paired read). Molecule “a” generates reads with associate a unique barcode with a unique barcode L_{A1-x}. No other molecule should have the same combination. If two molecules from the same construct have a common barcode, the data is discarded due to the ambiguity of the source molecule for those reads. Fragments b₁, b₂, c₁, c₂ etc. are identified by one read of the paired read with the barcode. The other read is used to make consensus sequence of internal regions of the molecule. The consensus sequence for each clone is compared with the desired sequence. The example shows results from well A1 in which clone x is correct, but clone y and z are incorrect. Similar results for each of the original constructs pooled together can be obtained in parallel from the sequencing results.

In step VI, FIG. 1C, the correct construct sequences can be amplified using a pair of primers in each well which have the unique tag sequences from the tag pair corresponding to the correct nucleic acid clone. Each clone can be amplified with the tagged pool as a template in individual wells. This allow for the generation of a plate of cloned constructs, each well containing a different desired sequence with each molecule having the correct sequence. As illustrated in FIG. 1C, the molecules in each well are in vitro clones of the original constructs, with flanking sequences corresponding to the barcode combination (K,L) used to amplify the clones having the correct predetermined sequence.

### EQUIVALENTS

The present invention provides among other things novel methods and devices for high-fidelity gene assembly. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

Reference is made to U.S. provisional application 61/637,750. filed Apr. 24, 2012 U.S. provisional application Ser. No. 61/638,187, filed Apr. 25, 2012, and U.S. provisional application Ser. No. 61/731,626, filed Nov. 30, 2012. All publications, patents and sequence database entries mentioned herein are hereby incorporated by reference in their entirety as if each individual publication or patent was specifically and individually indicated to be incorporated by reference.

1. A method of sorting nucleic acid molecules having a predetermined sequence, the method comprising:
(a) contacting a pool of nucleic acid molecules comprising at least two populations of nucleic acid molecules, each population of nucleic acid molecule having a unique nucleic acid sequence;
(b) tagging the 5′ end and the 3′ end of the nucleic acid molecules with an oligonucleotide tag sequence, wherein the oligonucleotide tag sequence comprises a unique nucleotide tag and a primer region;
(c) subjecting the nucleic acid molecules to a sequencing reaction from both ends to obtain a paired end read; and
(d) sorting the nucleic acid molecules having the predetermined sequence according to the identity of their corresponding unique nucleotide tags.

2. The method of claim 1 further comprising amplifying the nucleic acid molecules having the predetermined sequence.

3. The method of claim 2 further comprising amplifying the constructs having the predetermined sequence using primers complementary to the primer region.

4. The method of claim 1 further comprising pooling a plurality of nucleic acid molecules to form the pool of nucleic acid molecules, wherein each plurality of nucleic acid molecules comprises a population of nucleic acid sequences having the predetermined sequence and a population of nucleic acid sequences having a sequence different than the predetermined sequence.

5. The method of claim 4 further comprising assembling the plurality of nucleic acid molecules onto a solid support prior to pooling the plurality of nucleic acid molecules.

6. The method of claim 4 further comprising diluting the plurality of nucleic acid molecules prior to the step of pooling or after the step of pooling.

7. The method of claim 1 wherein in the step of contacting, the pool of nucleic acid molecules is normalized.

8. The method of claim 1 wherein in the step of tagging, the oligonucleotide tag sequences are ligated to the 5′ and 3′ end of the nucleic acid molecules.

9. The method of claim 1 wherein in the step of tagging, the oligonucleotide tag sequences are joined to the 5′ and 3′ end of the nucleic acid molecules by polymerase chain reaction.

10. The method of claim 1 wherein in the step of contacting, each nucleic acid molecule comprises a 5′ end common adaptor sequence and 3′ end common adaptor sequence and wherein the oligonucleotide tag sequence further comprises a common adaptor sequence.

11. The method of claim 1 wherein in the step of contacting, each population of nucleic acid molecules has a different desired nucleic acid sequence.

12. The method of claim 1 wherein in the step of tagging the unique oligonucleotide tag is a degenerate nucleotide sequence.
